(19) United States
(12) Patent Application Publication
(10) Pub. No.: US 2020/0201074 A1
(43) Pub. Date: Jun. 25, 2020

(54) SMART CONTACT LENS LOADED WITH DRUG

(71) Applicant: INTEROJO INC., Pyeongtaek-si (KR)

(72) Inventors: Gyen-Hei KIM, Gimhae-si (KR); Young YU, Seoul (KR); Si-Youl YOO, Suwon-si (KR); Hyo-Jin JEONG, Bucheon-si (KR); Hye-Ran NOH, Seoul (KR)

(21) Appl. No.: 16/379,613

(22) Filed: Apr. 9, 2019

(30) Foreign Application Priority Data
Dec. 19, 2018 (KR) 10-2018-0165052

(51) Int. Cl. G02C 7/04; A61F 9/00

(52) U.S. Cl. CPC G02C 7/049; A61F 9/0017

(57) ABSTRACT

A contact lens includes a hydrogel that includes a three-dimensional network of cross-linked polymer chains; and at least one ophthalmic drug embedded in the three-dimensional network of cross-linked polymer chains. In the contact lens, the hydrogel includes a first repeating unit derived from hydroxyethyl methacrylate and a second repeating unit derived from N-isopropylacrylamide. The hydrogel including the first and second repeating units is configured to swells when absorbing water such that, when the contact lens is placed in or contacts an aqueous solution having pH in a range between 5.80 and 8.03, the lower the pH of the aqueous solution within the range, the more the hydrogel absorbs water and swell and the more the contact lens releases the at least one ophthalmic drug into the aqueous solution.

### CROSS-REFERENCE TO RELATED APPLICATIONS

This application claims priority under 35 U.S.C. § 119 to Korean Patent Application No. 10-2018-0165052, filed on Dec. 19, 2018, in the Korean Intellectual Property Office, the disclosure of which is incorporated herein by reference in its entirety.

### TECHNICAL FIELD

The following disclosure relates to a smart contact lens loaded with a drug, and in particular, to a contact lens capable of loading a drug and having pH sensitivity, and a method of producing the same.

### BACKGROUND

Since liquid and ointment ophthalmic drugs for treating an eye disease are convenient to use and inexpensive, the ophthalmic drug market has been growing. Recently, various research such as adjustment of a lipid layer thickness, formation of a tear film, alleviation of an eye disease, and the like using a newly developed ophthalmic emulsion drug have been conducted.

However, although the ophthalmic drug is used frequently, since a liquid ophthalmic drug to be administered is mostly lost due to blinking of the eyelids or tear drainage from the eyes, the amount of liquid ophthalmic drug which actually reaches the cornea and exhibits its effect is as low as 5% or less. Therefore, repetitive administration of the liquid ophthalmic drug is required for treatment effectiveness.

Further, an ointment ophthalmic drug stays in the eyes for a relatively long time, such that the drug is more likely to be absorbed. However, discomfort is caused by a foreign body sensation due to a semi-solid form of ointment and visibility is significantly reduced after administration.

Therefore, the necessity for a development of an ophthalmic drug sustaining drug effectiveness, capable of controlling a release rate of the drug, and having an excellent biocompatibility has emerged. As an alternation of the existing administration manner, an interest in a drug-loaded contact lens which is produced by loading a drug in a contact lens used by 140 million or more people all over the world, can be wearable for a long period of time, and sustainably releases the drug, has increased.

As a drug-loaded contact lens of the related art, a therapeutic contact lens including a lens body having a spherical surface to be worn on the eye and a receiving portion formed in an inner surface in contact with the eye, receiving a drug of ointment or gel type, and having a recessed groove shape has been developed.

However, in such a drug-loaded contact lens, drug release is not controlled and the drug is released through an outer hole of the lens every time the eye blinks, such that the entire amount of drug is released in a very short time.

A soft contact lens made of hydroxyethyl methacrylate (HEMA) which is a main raw material of a contact lens has flexibility similar to the human cornea and property of a hydrogel which swells when in contact with water.

Studies have been attempted to develop a contact lens which loads a drug using a stimulus-sensitive hydrogel of which solubility or affinity for water changes due to stimuli such as light, a temperature, a pH, a chemical substance, and an electromagnetic field, as the hydrogel described above, and can control drug release by the stimulus.

However, in the drug-loaded contact lens using the hydrogel, it takes a long time for loading a drug and treatment is not effective due to a short drug release time. In particular, it is difficult to apply the drug-loaded contact lens to treatment for a chronic eye disease. Therefore, the ophthalmic drug still has a large market share.

Meanwhile, a major component of tears is water (98%), and phosphate, fat, albumin, globulin, sodium chloride, sodium carbonate, and the like are contained in tears. A pH of tears is 7.2 to 7.5. It is reported that the pH of tears changes within pH 5.2 to 8.6 depending on an eye disease, an emotional state, and an external environment change.

The disclosure of this section is to provide background of the invention. Applicant notes that this section may contain information available before this application. However, by providing this section, Applicant does not admit that any information contained in this section constitutes prior art.

### SUMMARY

One aspect of the invention provides a contact lens, which comprises: a hydrogel comprising a three-dimensional network of cross-linked polymer chains; and at least one ophthalmic drug embedded in the three-dimensional network of cross-linked polymer chains. In the contact lens, the hydrogel comprises a first repeating unit derived from hydroxyethyl methacrylate and a second repeating unit derived from N-isopropylacrylamide. The hydrogel comprising the first and second repeating units is configured to swells when absorbing water such that, when the contact lens is placed in or contacts an aqueous solution having pH in a range between 5.80 and 8.03, the lower the pH of the aqueous solution within the range, the more the hydrogel absorbs water and swell and the more the contact lens releases the at least one ophthalmic drug into the aqueous solution.

In the foregoing contact lens comprising the second repeating unit derived from N-isopropylacrylamide, the at least one ophthalmic drug may comprise hydroxypropyl methylcellulose. When being placed in an aqueous solution at pH 5.80, the contact lens may be configured to release the at least one ophthalmic drug such that it takes 3-4 hours from beginning to release 50% of the total amount of the at least one ophthalmic drug.

Another aspect of the invention provides a method of making the foregoing contact lens comprising the second repeating unit derived from N-isopropylacrylamide. The method comprises: mixing N-isopropylacrylamide, hydroxyethyl methacrylate and a crosslinking agent to provide a composition; injecting the composition into a contact lens mold to form the hydrogel therein; and subsequently loading the at least one ophthalmic drug such that the at least one ophthalmic drug is embedded in the three-dimensional network of cross-linked polymer chains. The at least one ophthalmic drug may comprise hydroxypropyl methylcellulose. When being placed in an aqueous solution at pH 5.80, the contact lens may be configured to release the at least one ophthalmic drug such that it takes 3-4 hours from beginning to release 50% of the total amount of the at least one ophthalmic drug.

Still another aspect of the invention provides a method comprising: providing the foregoing contact lens comprising the second repeating unit derived from N-isopropylacrylamide; and placing the contact lens in an eye of a subject in need of treatment with the at least one ophthalmic drug. It may take 3-4 hours to release 50% of the total amount of the at least one ophthalmic drug into the eye. The at least one ophthalmic drug may comprise hydroxypropyl methylcellulose.

One aspect of the invention provides a contact lens, which comprises: a hydrogel comprising a three-dimensional network of cross-linked polymer chains; and at least one ophthalmic drug embedded in the three-dimensional network of cross-linked polymer chains. In the contact lens, the hydrogel comprises a first repeating unit derived from hydroxyethyl methacrylate and a second repeating unit derived from N-vinylpyrrolidone. The hydrogel comprising the first and second repeating units is configured to swells when absorbing water such that, when the contact lens is placed in or contacts an aqueous solution having pH in a range between 5.80 and 8.03, the higher the pH of the aqueous solution within the range, the more the hydrogel absorbs water and swell and the more the contact lens releases the at least one ophthalmic drug into the aqueous solution.

In the foregoing contact lens comprising the second repeating unit derived from N-vinylpyrrolidone, the at least one ophthalmic drug may comprise hydroxypropyl methylcellulose. When being placed in an aqueous solution at pH 8.03, the contact lens may be configured to release the at least one ophthalmic drug such that it takes 3-4 hours from beginning to release 50% of the total amount of the at least one ophthalmic drug.

Another aspect of the invention provides a method of making the foregoing contact lens comprising the second repeating unit derived from N-vinylpyrrolidone. The method comprises: mixing N-vinylpyrrolidone, hydroxyethyl methacrylate and a crosslinking agent to provide a composition; injecting the composition into a contact lens mold to form the hydrogel therein; and subsequently loading the at least one ophthalmic drug such that the at least one ophthalmic drug is embedded in the three-dimensional network of cross-linked polymer chains. The at least one ophthalmic drug may comprise hydroxypropyl methylcellulose. When being placed in an aqueous solution at pH 8.03, the contact lens may be configured to release the at least one ophthalmic drug such that it takes 3-4 hours from beginning to release 50% of the total amount of the at least one ophthalmic drug.

Still another aspect of the invention provides a method comprising: providing the foregoing contact lens comprising the second repeating unit derived from N-vinylpyrrolidone; and placing the contact lens in an eye of a subject in need of treatment with the at least one ophthalmic drug. It may take 3-4 hours to release 50% of the total amount of the at least one ophthalmic drug into the eye. The at least one ophthalmic drug may comprise hydroxypropyl methylcellulose.

An embodiment of the present invention is directed to providing a smart contact lens having a high water content and biocompatibility, capable of loading a drug, and having pH sensitivity.

Another embodiment of the present invention is directed to providing a smart contact lens capable of preventing an eye disease and improving treatment effectiveness by controlling drug release depending on a change in an eye pH.

Still another embodiment of the present invention is directed to providing a smart contact lens releasing a drug effectively in a sustained manner and releasing the drug for a long period of time.

In one general aspect, a composition for producing a contact lens, contains: a vinyl amide which is any one selected from N-vinylpyrrolidone or N-isopropylacrylamide, or a mixture thereof; hydroxyethyl methacrylate; and a crosslinking agent.

A mixing ratio of the vinyl amide to the hydroxyethyl methacrylate may be 1:1 to 20 by weight.

The crosslinking agent may be one or two or more selected from the group consisting of ethylene glycol dimethacrylate, diethylene glycol dimethacrylate, trimethylol propanetriacrylate, trimethylolpropane trimethacrylate, N,N′-methylenebisacrylamide, N,N′-ethylenebisacrylamide, and N,N′-hexamethylenebisacrylamide.

In another general aspect, a contact lens produced by polymerizing the composition for producing a contact lens is provided.

The contact lens may be a pH sensitive contact lens.

The contact lens may be a contact lens for delivering a drug.

The contact lens may be produced by polymerizing a composition for producing a contact lens containing N-vinylpyrrolidone, hydroxyethyl methacrylate, and a crosslinking agent.

The contact lens may be a pH sensitive contact lens loaded with a drug and further releasing the drug when a pH increases.

The contact lens may be produced by polymerizing a composition for producing a contact lens containing N-isopropylacrylamide, hydroxyethyl methacrylate, and a crosslinking agent.

The contact lens may be a pH sensitive contact lens loaded with a drug and further releasing the drug when a pH decreases.

The contact lens may release 50 wt % of the loaded drug within 1 to 6 hours and release 80 wt % or more of the loaded drug within 12 hours.

In still another general aspect, a method of producing a contact lens, includes: mixing a composition for producing a contact lens containing a vinyl amide which is any one selected from N-vinylpyrrolidone or N-isopropylacrylamide, or a mixture thereof, hydroxyethyl methacrylate, and a crosslinking agent; and injecting the mixed composition into a contact lens mold and polymerizing the mixed composition.

The method may further include loading a drug after the polymerization.

Other features and aspects will be apparent from the following detailed description, the drawings, and the claims.

### BRIEF DESCRIPTION OF THE DRAWINGS

FIGS. 1A and 1B are graphs showing light transmittance of a contact lens according to an embodiment of the present invention.

FIGS. 2A and 2B are graphs showing a water content of a contact lens according to an embodiment of the present invention.

FIGS. 3A to 3D are graphs showing drug release over time of a contact lens according to an embodiment of the present invention.

FIGS. 4A to 4D are graphs showing drug release over time of a contact lens according to an embodiment of the present invention.

FIG. 5 is a graph showing drug release over time of a contact lens according to Comparative Example.

FIGS. 6A and 6B are graphs showing drug release depending on a pH of a contact lens according to an embodiment of the present invention.

FIG. 7 is a graph showing a drug release depending on a periodic change in a pH of a contact lens according to an embodiment of the present invention.

### DETAILED DESCRIPTION OF EMBODIMENTS

The advantages, features and aspects of the present invention will become apparent from the following description of the embodiments with reference to the accompanying drawings, which is set forth hereinafter. The present invention may, however, be embodied in different forms and should not be construed as limited to the embodiments set forth herein. Rather, these embodiments are provided so that this disclosure will be thorough and complete, and will fully convey the scope of the present invention to those skilled in the art. The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Hereinafter, embodiments will be described in detail with reference to the accompanying drawings. However, the following Examples and Comparative Examples are only one reference example for describing the present invention in detail, the present invention is not limited thereto, and may be implemented in various forms.

Unless otherwise defined, all terms of technical and scientific terms used herein have the same meaning as commonly understood by one of person skilled in the art to which the present invention belongs. Terms used in the present invention are used only in order to describe an embodiment rather than limiting the present invention.

In addition, a hydrogel in the present disclosure means a solid material containing a hydrophilic polymer which swells with water as a solvent and means that it is not actually deformed in a normal state due to having a high-viscosity and has no fluidity due to having a three-dimensionally physical or chemical crosslinking bond.

In addition, singular forms used in the specification and the appended claims may be intended to include plural forms unless interpreted otherwise in context.

One embodiment of the present invention provides a composition for producing a contact lens for producing a smart contact lens capable of controlling drug release depending on a change in a pH. The composition for producing a contact lens includes a vinyl amide which is any one selected from N-vinylpyrrolidone or N-isopropylacrylamide, or a mixture thereof, hydroxyethyl methacrylate, and a crosslinking agent.

The composition for producing a contact lens according to embodiments of the present invention contains the vinyl amide and the hydroxyethyl methacrylate as a monomer, and the crosslinking agent, such that the produced contact lens has hydrogel properties, may control drug release depending on a change in a pH, releases a drug effectively in a sustained manner, and releases the drug for a long period of time.

In general, a pH of tears is within a range of 7.2 to 7.5 and the pH of tears changes depending on an eye disease, an emotional state, and an external environment change within a range of 5.2 to 8.6. The contact lens according to embodiments of the present invention may control drug release even within a narrow pH range of a weak acidic or a weak base, thus it is possible to prevent an eye disease and improve treatment effectiveness.

The vinyl amide may be any one selected from N-vinylpyrrolidone or N-isopropylacrylamide, or a mixture thereof.

A hydrogel contact lens having a three-dimensional network structure may be produced by polymerizing the vinyl amide with the hydroxyethyl methacrylate and the crosslinking agent, and enable the contact lens to be excellent in biocompatibility, optical property, swellability, and wearability.

Specifically, in a case where the composition contains N-vinylpyrrolidone as the vinyl amide, a strong hydrogen bond is formed between a carbonyl functional group in the N-vinylpyrrolidone and a hydroxy functional group in the hydroxyethyl methacrylate.

In addition, in a case where the composition contains N-isopropylacrylamide as the vinyl amide, a strong hydrogen bond is formed between an amide functional group in the molecule and a hydroxy functional group in the hydroxyethyl methacrylate. Therefore, the hydrogel contact lens has a more stable three-dimensional network structure and a high crosslinking density.

Accordingly, the contact lens has a high water content due to an improved water-swellable property, prevents dry eyes, and does not cause discomfort even for long wear.

In particular, in a case where the contact lens is used as a contact lens for delivering a drug, a crosslinking density increases according to a strong hydrogen bond between the vinyl amide and the hydroxyethyl methacrylate, such that the contact lens for delivering a drug may control an initial release amount of loaded drug and release the loaded drug in a sustained manner.

In this case, it is possible to effectively sustain treatment without eye irritation and a foreign body sensation caused by a large amount of loaded drug mostly released in the initial stage in the contact lens for delivering a drug of the related art.

In an aspect of the present invention, a content of the vinyl amide in the monomer composition containing the vinyl amide and the hydroxyethyl methacrylate may be 1 to 50 wt %, preferably 2 to 45 wt %, and more preferably 3 to 40 wt %, and a content of the hydroxyethyl methacrylate in the composition may be 50 to 99 wt %, preferably 55 to 97 wt %, and more preferably 60 to 95 wt %, but is not limited thereto.

In an aspect of the present invention, a mixing ratio of the vinyl amide to the hydroxyethyl methacrylate in the composition for producing a contact lens may be 1:1 to 20 by weight, preferably 1:1 to 15 by weight, and more preferably 1:1 to 10 by weight. The vinyl amide and the hydroxyethyl methacrylate are mixed within the mixing ratio range described above, such that a contact lens to be produced is excellent in pH sensitivity even within a narrow pH range and wearability.

In particular, in a case where a contact lens produced by a composition for a contact lens containing vinyl amide and hydroxyethyl methacrylate having the mixing ratio range described above is used as a drug-loaded contact lens, the time required for loading a drug is short, the drug is may be released in a sustained manner after wearing the drug-loaded contact lens, and drug release time is long.

Therefore, ease of production and treatment effectiveness of the drug-loaded contact lens are significantly improved and it is effective to treat and prevent an eye disease caused by a change in a pH even while a user is wearing a contact lens for vision correction.

In an aspect of the present invention, the crosslinking agent may be one or two or more selected from the group consisting of ethylene glycol dimethacrylate, diethylene glycol dimethacrylate, trimethylol propanetriacrylate, trimethylolpropane trimethacrylate, N,N′-methylenebisacrylamide, N,N′-ethylenebisacrylamide, and N,N′-hexamethylenebisacrylamide.

A content of the crosslinking agent may be 0.001 to 0.01 wt %, and preferably 0.001 to 0.005 wt %, with respect to the hydroxyethyl methacrylate, but is not limited thereto.

By using the crosslinking agent, a main chain of a polymer composed of a vinyl amide monomer and a hydroxyethyl methacrylate monomer is crosslinked, thus a hydrogel having a three-dimensional network structure may be formed.

In particular, the vinyl amide and the hydroxyethyl methacrylate are used together with the crosslinking agent having the content range described above, such that a hydrogel is easily formed. A contact lens to be produced has flexibility conform to the eye cornea and an appropriate strength, and also effectively loads a drug and releases the drug in a sustained manner.

In addition, embodiments the present invention relate to a contact lens produced by polymerizing the composition for producing a contact lens.

The contact lens according to embodiments of the present invention may be produced by polymerizing a composition for producing a contact lens containing a vinyl amide monomer which is any one selected from N-vinylpyrrolidone or N-isopropylacrylamide, or a mixture thereof, a hydroxyethyl methacrylate monomer, and a crosslinking agent by using polymerization methods such as a photopolymerization method, a thermal polymerization method, and the like.

Depending on vinyl amide in a composition for producing a contact lens, a contact lens may be a contact lens of a first aspect produced by polymerizing a composition for producing a contact lens containing N-vinylpyrrolidone, hydroxyethyl methacrylate, and a crosslinking agent; a contact lens of a second aspect produced by polymerizing a composition for producing a contact lens containing N-isopropylacrylamide, hydroxyethyl methacrylate, and a crosslinking agent; and a contact lens of a third aspect produced by polymerizing a composition for producing a contact lens containing N-vinylpyrrolidone, N-isopropylacrylamide, hydroxyethyl methacrylate, and a crosslinking agent.

The contact lens according to an aspect of the present invention includes the vinyl amide, the hydroxyethyl methacrylate monomer, and the crosslinking agent, such that the contact lens has an excellent biocompatibility, optical property, swellability, and wearability.

Specifically, a hydrogel contact lens having a crosslinked hydrogel property may be produced by using the vinyl amide, the hydroxyethyl methacrylate, and the crosslinking agent together, and a strong hydrogen bond is formed between the vinyl amide and the hydroxyethyl methacrylate. Therefore, the three-dimensional network structure is more stabilized and the crosslinking density is increased.

Accordingly, the contact lens has a high water content, prevents dry eyes, and does not cause discomfort even for long wear.

In an aspect of the present invention, the contact lens may be a pH sensitive contact lens in which a swelling rate varies depending on a pH, in this case, the pH may be 5.0 to 9.0. The pH is a transition pH. The transition pH may be measured indirectly by measuring a swelling rate of a hydrogel contact lens, and an inflection point of a slope within a range in which the swelling rate is varied may be determined as the transition pH.

As the contact lens according to an aspect of the present invention, the contact lens of the first aspect produced by polymerizing a composition for producing a contact lens containing N-vinylpyrrolidone, hydroxyethyl methacrylate, and a crosslinking agent swells when a pH increases, specifically, the transition pH may be pH 5.80 to 8.03, and more specifically, pH 7.20 to 8.03.

In addition, the contact lens of the second aspect produced by polymerizing a composition for producing a contact lens containing N-isopropylacrylamide, hydroxyethyl methacrylate, and a crosslinking agent swells when a pH decreases, specifically, the transition pH may be pH 5.80 to 8.03, and more specifically, pH 5.80 to 7.20.

In addition, the contact lens of the third aspect produced by polymerizing a composition for producing a contact lens containing N-vinylpyrrolidone, N-isopropylacrylamide, hydroxyethyl methacrylate, and a crosslinking agent may adjust a shrinkage and swelling behavior of the contact lens depending on a pH according to a mixing ratio of the vinyl amide in the composition, that is, a mixing ratio of N-vinylpyrrolidone and N-isopropylacrylamide. Specifically, for example, in a case where N-vinylpyrrolidone is 60 wt % or more based on the total weight of the vinyl amide, the contact lens swells when a pH increases and shrinks when a pH decreases. In a case where N-isopropylacrylamide is more than 60 wt % based on the total weight of the vinyl amide, the contact lens shrinks when a pH increases and swells when a pH decreases.

In an aspect of the present invention, the contact lens may be a contact lens produced by polymerizing the hydrogel containing the composition for producing a contact lens as a dispersed phase.

Specifically, a continuous phase of the hydrogel is generally used in the related art to which the present invention belongs, any polymer can be used as long as it is able to form a hydrogel, for example, may be a hydrophilic polymer with no pH sensitivity, but is not limited thereto.

More specifically, for example, the composition for producing a contact lens of the first, second, or third aspect may be dispersed in a continuous phase of the hydrophilic polymer, and a pH sensitive contact lens having hydrogel properties may be produced by polymerizing the composition.

Since the contact lens according to an aspect of the present invention has pH sensitivity within a relatively narrow pH range, it is possible to be applied to various fields required for sensitivity depending on a minute change in a pH such as a contact lens which is sensitive to a change in a pH of tears.

According to an aspect of the present invention, the contact lens may be a contact lens for delivering a drug. In the case of a contact lens for delivering a drug of the related art, as the drug is released every time the eye blinks, the entire amount of drug loaded in the contact lens is released in a very short time, resulting in ineffective treatment. On the other hand, the contact lens according to embodiments of the present invention may release a drug in a sustained manner and realize long-term drug release, resulting in treatment effectiveness.

In a case where a sensitive hydrogel of which swellability changes due to stimuli such as a pH and a temperature is applied to a contact lens for delivering a drug, as a large amount of drug is released due to rapid swelling of the contact lens due to a stimulus, resulting in side effects of a drug and ineffective treatment. On the other hand, the contact lens according to embodiments of the present invention decreases an initial release amount of drug as a crosslinking density increases according to a strong hydrogen bond between the vinyl amide and the hydroxyethyl methacrylate, such that the contact lens for delivering a drug decreases an initial release amount of drug and may release the drug in a sustained manner.

The contact lens for delivering a drug according to the first aspect of the present invention may be a pH sensitive contact lens which further releases the drug when a pH increases. The contact lens swells at a pH of 5.0 to 9.0, specifically, a pH of 5.80 to 8.03, and more specifically, a pH 7.20 to 8.03 and further releases the drug.

The contact lens for delivering a drug according to the second aspect of the present invention may be a pH sensitive contact lens which further releases the drug when a pH decreases. The contact lens swells at a pH of 5.0 to 9.0, specifically, a pH of 5.80 to 8.03, and more specifically, a pH 5.80 to 7.20 and further releases the drug.

The contact lens for delivering a drug according to the third aspect of the present invention may control a drug release behavior depending on a mixing ratio of vinyl amide in the composition, that is, a mixing ratio of N-vinylpyrrolidone and N-isopropylacrylamide and may be a pH sensitive contact lens which swells at pH 5.0 to 9.0 and further releases a drug.

Specifically, for example, in a case where N-vinylpyrrolidone is 60 wt % or more based on a total weight of the vinyl amide, the contact lens further releases a drug when the pH increases within the pH range described above.

In addition, in a case where N-isopropylacrylamide is 60 wt % or more based on a total weight of the vinyl amide, the contact lens further releases a drug when the pH decreases within the pH range described above.

In an aspect of the present invention, the contact lens may release 50 wt % of the loaded drug within 1 to 6 hours and release 80 wt % or more of the loaded drug within 12 hours. More specifically, the contact lens may release 50 wt % of the loaded drug within 1 to 4 hours and release 80 wt % or more of the loaded drug within 10 hours, but is not limited thereto.

In the case of the contact lens for delivering a drug of the related art, the drug is rapidly released after wearing the contact lens, and thus 80 wt % or more of the loaded drug is excessively released within 6 hours. Thus, there may be side effects of a drug, a foreign body sensation, and ineffective treatment.

However, in a contact lens according to embodiments of the present invention, an initial release amount of drug is low and the drug is released in a sustained manner for a long period of time. Therefore, the drug loaded in the contact lens is released for a relatively long time within the range described above and may be consistently transferred to a surface and a tissue of the eye. That is, the drug is sustainably released in vivo for absorption available time, thus treatment effectiveness can be maximized.

In addition, since the contact lens is sensitive to a minute change in an eye pH due to an external environment or an eye disease and releases a drug, the contact lens effectively prevents an eye disease. This is still in effect even while a user is wearing a contact lens for vision correction, thus prevention of an eye disease and treatment effectiveness can be maximized through a contact lens for delivering a drug.

In an aspect of the present invention, a water content of the contact lens may be 25 to 70%, specifically, 30 to 70%, and more specifically, 35 to 70%, at this time, the water content refers to a weight ratio of the swollen contact lens based on the weight of the dried contact lens.

The contact lens according to embodiments of the present invention has an excellent biocompatibility, swellability in absorbed water, the water content within the range described above, the contact lens prevents dry eyes and has excellent in maintaining oxygen permeability.

Accordingly, in the contact lens according to embodiments of the present invention may be wearable for a long period of time without the user's discomfort, eye irritation, and a wearing problem of the contact lens against the cornea when a user wears the contact lens.

In addition, light transmittance of the contact lens may be 70 to 98%, specifically, 75 to 98%, and more specifically, 80 to 98%. Having the excellent light transmittance within the range described above, the contact lens has an excellent optical transparent.

One embodiment of the prevent invention provides a method of producing a contact lens, including: mixing a composition for producing a contact lens containing a vinyl amide which is any one selected from N-vinylpyrrolidone or N-isopropylacrylamide, or a mixture thereof, hydroxyethyl methacrylate, and a crosslinking agent; and injecting the mixed composition into a contact lens mold and polymerizing the mixed composition.

In the mixing of the composition, the composition for producing a contact lens containing the vinyl amide, the hydroxyethyl methacrylate, and the crosslinking agent may be stirred for 10 minutes to 1 hour.

As a monomer in the composition for producing a contact lens, a content of the vinyl amide may be 1 to 50 wt %, preferably 2 to 45 wt %, and more preferably 3 to 40 wt %, and a content of the hydroxyethyl methacrylate may be 50 to 99 wt %, preferably 55 to 97 wt %, and more preferably 60 to 95 wt %, but is not limited thereto.

In addition, a mixing ratio of the vinyl amide to the hydroxyethyl methacrylate may be 1:1 to 20 by weight, preferably 1:1 to 15 by weight, and more preferably 1:1 to 10 by weight.

A content of the crosslinking agent may be 0.001 to 0.01 wt %, and preferably 0.001 to 0.005 wt % with respect to the hydroxyethyl methacrylate, but is not limited thereto.

In addition, the composition may contain an initiator. Examples of the initiator may be, for example, a photoinitiator, a thermal initiator, and the like which are generally used in the related field. A content of the initiator may be 0.001 to 0.01 wt %, and preferably 0.001 to 0.005 wt % with respect to the content of hydroxyethyl methacrylate, but is not limited thereto.

By using the content and the mixing ratio of the composition of the range described above, the contact lens produced after polymerization maintains an excellent transparency without decrease in light transmittance. When this contact lens loads a drug and is used as a contact lens for delivering a drug, it is possible to load a drug in a short time, resulting in ease of production.

Subsequently, in the polymerization, the mixed composition for producing a contact lens is injected into an upper mold and a lower mold for a contact lens, and then the injected composition is polymerized to produce a contact lens. The polymerization is not limited to the method described above, but may be, for concrete examples, a method in which the injected composition for a contact lens may be subjected to thermal treatment at 60 to 130° C., and specifically, 80 to 110° C. for 1 to 6 hours.

The contact lens produced by the production method may control drug release depending on a change in a pH, releases a drug effectively in a sustained manner, and releases the drug for a long period of time.

In particular, since drug release may be controlled within in a narrow range of a pH change, it is possible to prevent an eye disease and improve treatment effectiveness by wearing of the contact lens.

In an aspect of the present invention, the method may further include loading a drug after the polymerization.

The drug described above may be any one or a mixture of two or more selected from eye drops, an antibiotic, an anti-inflammatory, an antihistaminic agent, an antiviral agent, an anticancer drug, an anesthetic, a cycloplegia, an iridodilator, a lubricant, a decongestant, an angiotonic, an immunosuppressant, an immunomodulatory agent, and a glaucoma drug, but is not limited thereto.

As the drug described above, hydroxypropyl methylcellulose (HPMC) eye drops may be preferably used. HPMC is mainly used in artificial tears eye drops, has an excellent moisturizing effect, hardly irritates the eye, and makes proliferation of cells existing in the surface of the cornea more active so that the cornea protection is improved, which is effective in treating dry eyes. In particular, the HPMC eye drops may be used while wearing a contact lens.

The drug loading method may be a method generally used in the related field, for example, a method of loading a drug by using an impregnation method, but is not limited thereto. Specifically, the contact lens produced by polymerization is washed using distilled water, dried thoroughly, and then immersed in an aqueous solution containing a drug for 0.5 to 6 hours, and specifically, 1 to 3 hours, thereby loading the drug in the contact lens. At this time, a content of the drug in the aqueous solution may be 0.01 to 5 wt %, and specifically, 0.01 to 3 wt %.

Hereinafter, the present invention will be described in more detail on the basis of Examples and Comparative Examples. However, the following Examples and Comparative Examples are only examples for describing the present invention in more detail, and the present invention is not limited by Examples and Comparative Examples.

Physical properties of a contact lens were measured by the following methods.

### (1) Light Transmittance

UV-B, UV-A, and visible light transmittances were measured by using a light transmittance meter (CL-100 (TM-1), TOPCON, Japan).

### (2) Water Content

A water content (%) was obtained by measuring a weight of a dried contact lens (Wdry) and a weight of a swollen contact lens (Wswell) after the respective contact lenses were impregnated in each aqueous solutions of pH 5.80, 6.45, 7.20, and 8.03 and evaluated the following Expression.

Water content (%)=(Wswell−Wdry)/Wdry×100

### EXAMPLE 1

0.95 g of 2-hydroxyethyl methacrylate (HEMA, manufactured by Aldrich), 0.05 g of N-vinylpyrrolidone (VP, manufactured by Daejung Co., Ltd.), 0.01 μL of ethylene glycol dimethacrylate (EGDMA, manufactured by Aldrich), and 0.01 μL of azobisisobutyronitrile (AIBN, manufactured by Aldrich) are stirred for 30 minutes, thereby preparing a composition for producing a contact lens.

Then, 80 μL of the prepared composition was injected into a mold for a contact lens, transferred to an oven heated to 100° C. and polymerized for 4 hours, and then the polymerized contact lens was separated from the mold.

Next, the polymerized contact lens was washed using distilled water for 5 minutes, hydrated in 0.9 wt % of sodium chloride aqueous solution (physiological saline) for 24 hours, removed from the solution, and dried at 25° C., thereby producing a contact lens.

### EXAMPLE 2

A contact lens was produced in the same manner as in Example 1 except that 0.90 g of 2-hydroxyethyl methacrylate (HEMA, manufactured by Aldrich) and 0.10 g of N-vinylpyrrolidone (VP, manufactured by Daejung Co., Ltd.) were used in the composition for producing a contact lens.

### EXAMPLE 3

A contact lens was produced in the same manner as in Example 1 except that 0.85 g of 2-hydroxyethyl methacrylate (HEMA, manufactured by Aldrich) and 0.15 g of N-vinylpyrrolidone (VP, manufactured by Daejung Co., Ltd.) were used in the composition for producing a contact lens.

### EXAMPLE 4

A contact lens was produced in the same manner as in Example 1 except that 0.80 g of 2-hydroxyethyl methacrylate (HEMA, manufactured by Aldrich) and 0.20 g of N-vinylpyrrolidone (VP, manufactured by Daejung Co., Ltd.) were used in the composition for producing a contact lens.

### EXAMPLE 5

0.95 g of 2-hydroxyethyl methacrylate (HEMA, manufactured by Aldrich), 0.05 g of N-isopropylacrylamide (NIPAAm, manufactured by Aldrich), 0.01 μL of methylenebisacrylamide (MBA, manufactured by Aldrich), and 0.01 μL of azobisisobutyronitrile (AIBN, manufactured by Aldrich) are stirred for 30 minutes, thereby preparing a composition for producing a contact lens.

Then, 80 μL of the prepared composition was injected into a mold for a contact lens, transferred to an oven heated to 100° C. and polymerized for 4 hours, and then the polymerized contact lens was separated from the mold.

Next, the polymerized contact lens was washed using distilled water for 5 minutes, hydrated in 0.9 wt % of sodium chloride aqueous solution (physiological saline) for 24 hours, removed from the solution, and dried at 25° C., thereby producing a contact lens.

### EXAMPLE 6

A contact lens was produced in the same manner as in Example 5 except that 0.90 g of 2-hydroxyethyl methacrylate (HEMA, manufactured by Aldrich) and 0.10 g of N-isopropylacrylamide (NIPAAm, manufactured by Aldrich) were used in the composition for producing a contact lens.

### EXAMPLE 7

A contact lens was produced in the same manner as in Example 5 except that 0.85 g of 2-hydroxyethyl methacrylate (HEMA, manufactured by Aldrich) and 0.15 g of N-isopropylacrylamide (NIPAAm, manufactured by Aldrich) were used in the composition for producing a contact lens.

### EXAMPLE 8

A contact lens was produced in the same manner as in Example 5 except that 0.80 g of 2-hydroxyethyl methacrylate (HEMA, manufactured by Aldrich) and 0.20 g of N-isopropylacrylamide (NIPAAm, manufactured by Aldrich) were used in the composition for producing a contact lens.

### COMPARATIVE EXAMPLE 1

1 g of 2-hydroxyethyl methacrylate (HEMA, manufactured by Aldrich), 0.01 μL of ethylene glycol dimethacrylate (EGDMA, manufactured by Aldrich), and 0.01 μL of azobisisobutyronitrile (AIBN, manufactured by Aldrich) are stirred for 30 minutes, thereby preparing a composition for producing a contact lens.

Then, 80 μL of the prepared composition was injected into a mold for a contact lens, transferred to an oven heated to 100° C. and polymerized for 4 hours, and then the polymerized contact lens was separated from the mold.

Next, the polymerized contact lens was washed using distilled water for 5 minutes, hydrated in 0.9 wt % of sodium chloride aqueous solution (physiological saline) for 24 hours, removed from the solution, and dried at 25° C., thereby producing a contact lens.

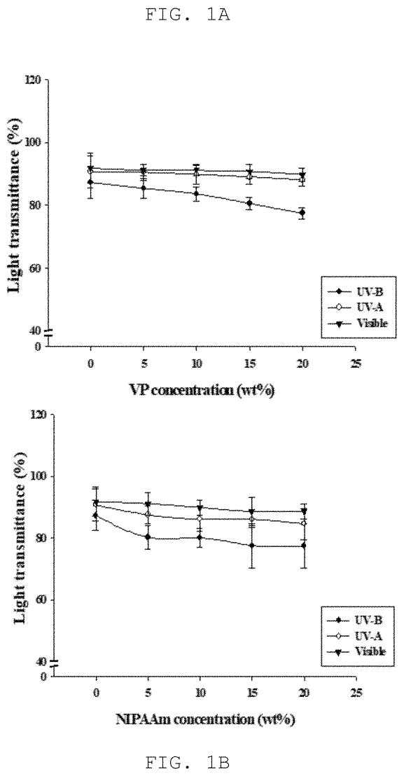

As a result, as shown in FIGS. 1A and 1B, the contact lenses of Examples 1 to 8 had an excellent light transmittance and it was confirmed that the contact lenses were transparent when observed with the naked eye.

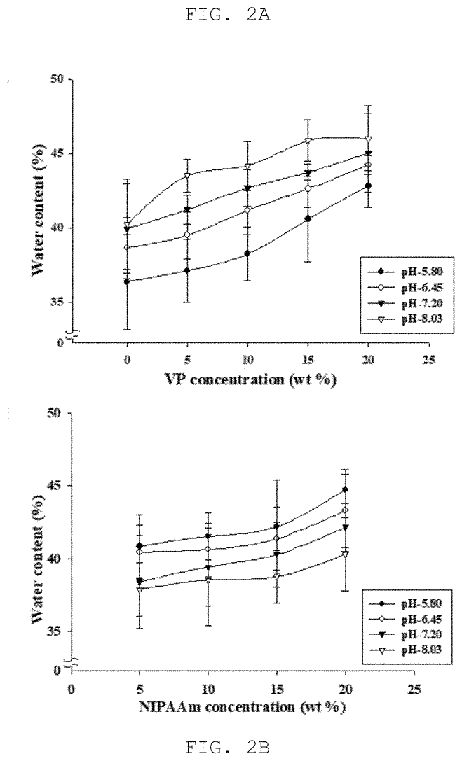

In addition, as shown in FIG. 2A, it was confirmed that as pH increases, the water contents of the contact lenses of Examples 1 to 4 were increased, and as shown in FIG. 2B, it was confirmed that as pH decreases, the water contents of the contact lenses of Examples 5 to 8 were increased.

### EXPERIMENTAL EXAMPLE 1

### Evaluation of Drug Release Property of Contact Lens

To evaluate drug release of the contact lenses produced in Examples 1 to 8 and Comparative Example 1, hydroxypropyl methylcellulose (HPMC, Grade 2910, manufactured by Aldrich) eye drops were used as a drug. The produced contact lenses were immersed in an aqueous solution of 100 mL in which a content of HPMC is 1 wt % for 3 hours and removed from the solution, and then dried at 25° C. The contact lenses loaded with the eye drops were put into 0.2 mL of phosphate buffered saline (PBS) solutions (manufactured by Amresco) of pH 5.80, 6.45, 7.20 and 8.03 prepared in 24-well plate, respectively, and in-vitro release experiment over time had been conducted at 25° C. for 12 hours. Absorbance of the released eye drops was measured using a UV-Spectrophotometer (Mega Array, manufactured by Scinco co., Ltd, Korea) at a wavelength of 290 nm and the amount of released eye drops which is converted was calculated.

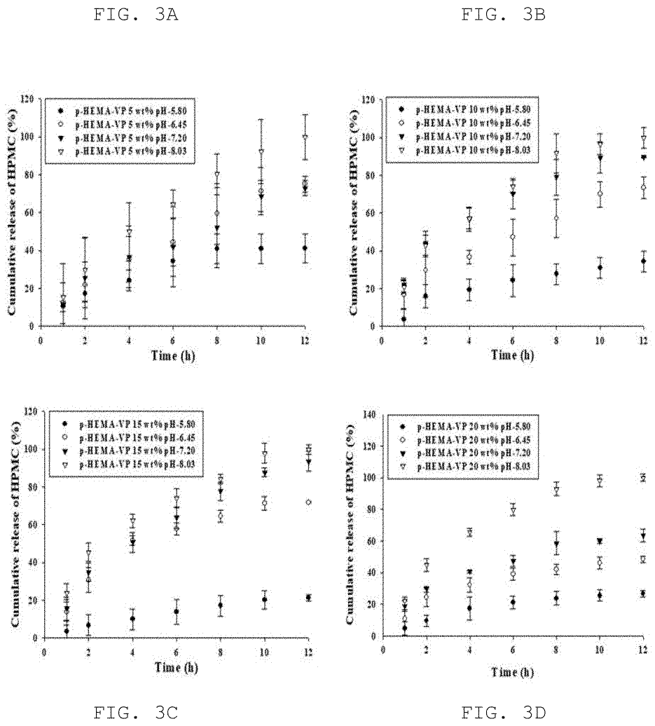

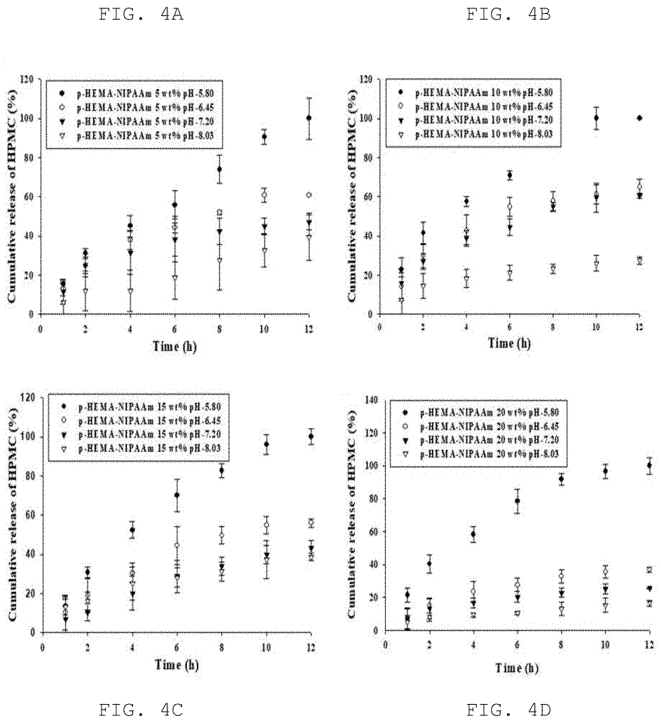

As a result, as shown in FIGS. 3A to 3D, it was confirmed that as pH increases, a release amount of a drug was increased in Examples 1 to 4, and as shown in FIGS. 4A to 4D, it was confirmed that as pH decreases, a release amount of a drug was increased in Examples 5 to 8.

In addition, it was confirmed that 50 wt % of the loaded drug was released within 3 to 4 hours and 80 wt % or more of the loaded drug was released within 8 hours at pH 8.03 in Examples 1 to 4, and 50 wt % of the loaded drug was released within 3 to 4 hours and 80 wt % or more of the loaded drug was released within 9 hours at pH 5.80 in Examples 5 to 8.

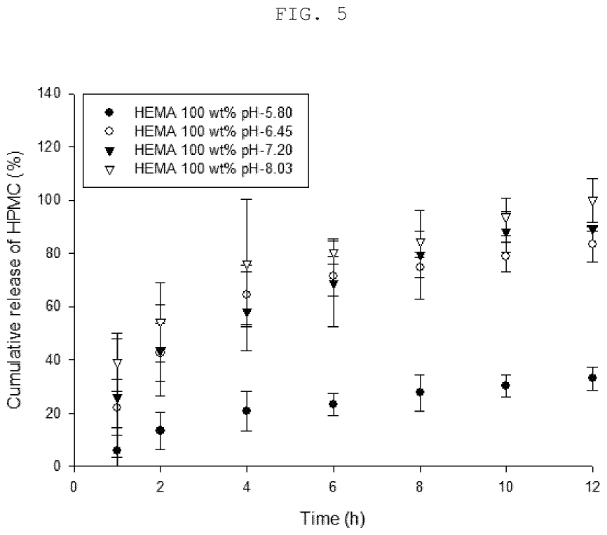

Meanwhile, as shown in FIG. 5, it was confirmed that in Comparative Example 1, as pH increases, a release amount of a drug was increased similarly to the behaviors in Examples 1 to 4, but 50 wt % of the loaded drug was released within 2 hours and 80 wt % or more of the loaded drug was released within 6 hours at pH 8.03. Accordingly, it was confirmed that the contact lens of Comparative Example 1 rapidly released the drug in the initial stage and release complete time was short.

That is, it was confirmed that since the contact lens according to Examples of the present invention has pH sensitivity, it is possible to control drug release depending on a change in a pH, control an initial release amount of loaded drug to be low, and release the drug in a sustained manner.

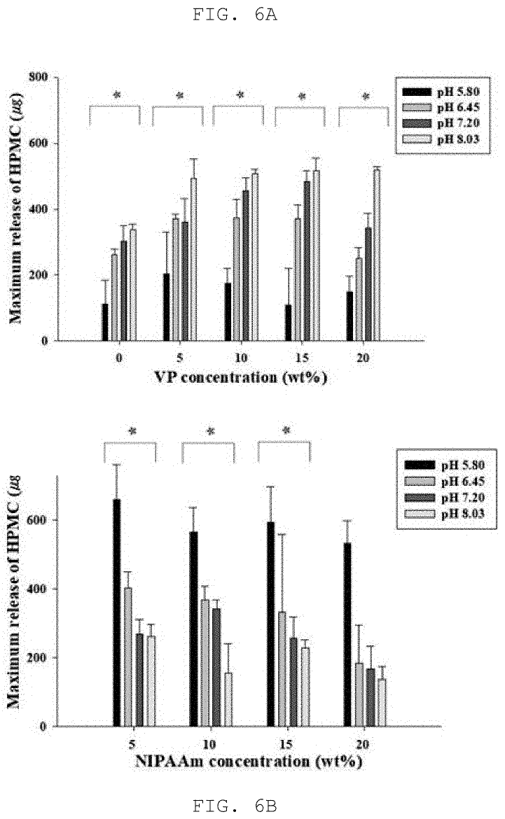

In addition, it was confirmed from the results as shown in FIGS. 6A and 6B that the maximum release amount of the drug and the cumulative release amount of the drug depending on a pH are high in Examples 1 to 8.

### EXPERIMENTAL EXAMPLE 2

### Evaluation of Drug Release Property of Contact Lens Depending on Periodic Change in pH

The contact lenses of Examples 2 and 6, and Comparative Example 1 were immersed in an aqueous solution of 100 mL in which a content of HPMC is 1 wt % for 3 hours and removed from the solution, and then dried at 25° C. Each 0.2 mL of phosphate buffered saline (PBS) solutions of pH 5.80 and 8.03 was prepared in 24-well plate, and in-vitro release experiment had been conducted by alternately immersing the contact lens loaded with the drug in the PBS solutions of pH 5.80 and 8.03 for 30 minutes. Absorbance of the released HPMC was measured using a UV-Spectrophotometer (Mega Array, manufactured by Scinco co., Ltd, Korea) at a wavelength of 290 nm and the amount of released HPMC which is converted was calculated.

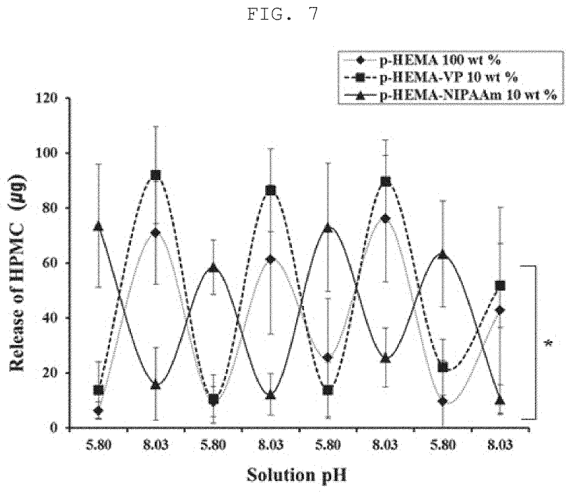

As a result, as illustrated in FIG. 7, it was confirmed that the contact lenses of Examples 2 and 6 show a consistent pH sensitivity, release rate, and release amount in spite of a repeated change in a pH, such that a release aspect is consistently maintained.

That is, it was confirmed that the contact lens according to an embodiment of the present invention has pH sensitivity, may release the drug in a sustained manner for a long period of time, and maintains an appropriate release amount of drug without rapidly releasing the drug when the contact lens releases the drug. Accordingly, it was confirmed that a drug stays in the eyes for a relatively long time so that the drug is more likely to be absorbed.

The composition for producing a contact lens according to an embodiment of the present invention may have an excellent transparency, water content, and biocompatibility and may be used for producing a smart contact lens which is sensitive to a pH change.

In addition, the smart contact lens according to an embodiment of the present invention may easily load a drug and may have pH sensitivity so that the drug may be released depending on a change in an eye pH.

Further, the smart contact lens according to an embodiment of the present invention may release a drug in a sustained manner and may be excellent in treatment effectiveness significantly due to a long drug release time.

Further, the smart contact lens of according to an embodiment of the present invention may prevent and treat an eye disease even while a user is wearing a contact lens for vision correction without a repetitive administration of eye drops, thereby providing excellent user convenience.

What is claimed is:

1. A contact lens comprising:
a hydrogel comprising a three-dimensional network of cross-linked polymer chains; and
at least one ophthalmic drug embedded in the three-dimensional network of cross-linked polymer chains,
wherein the hydrogel comprises a first repeating unit derived from hydroxyethyl methacrylate and a second repeating unit derived from N-isopropylacrylamide,
wherein the hydrogel comprising the first and second repeating units is configured to swells when absorbing water such that, when the contact lens contacts an aqueous solution having pH in a range between 5.80 and 8.03, the lower the pH of the aqueous solution within the range, the more the hydrogel absorbs water and swell and the more the contact lens releases the at least one ophthalmic drug into the aqueous solution.

2. The contact lens of claim 1, wherein the at least one ophthalmic drug comprises hydroxypropyl methylcellulose.

3. The contact lens of claim 1, wherein, when being placed in an aqueous solution at pH 5.80, the contact lens is configured to release the at least one ophthalmic drug such that it takes 3-4 hours from beginning to release 50% of the total amount of the at least one ophthalmic drug.

4. A method of making the contact lens of claim 1, the method comprising:
mixing N-isopropylacrylamide, hydroxyethyl methacrylate and a crosslinking agent to provide a composition;
injecting the composition into a contact lens mold to form the hydrogel therein; and
subsequently loading the at least one ophthalmic drug such that the at least one ophthalmic drug is embedded in the three-dimensional network of cross-linked polymer chains.

5. The method of claim 4, wherein the at least one ophthalmic drug comprises hydroxypropyl methylcellulose.

6. The method of claim 4, wherein, when being placed in an aqueous solution at pH 5.80, the contact lens is configured to release the at least one ophthalmic drug such that it takes 3-4 hours from beginning to release 50% of the total amount of the at least one ophthalmic drug.

7. A method comprising:
providing the contact lens of claim 1; and
placing the contact lens in an eye of a subject in need of treatment with the at least one ophthalmic drug.

8. The method of claim 7, wherein it takes 3-4 hours to release 50% of the total amount of the at least one ophthalmic drug into the eye.

9. A method comprising:
providing the contact lens of claim 1; and
placing the contact lens in an eye of a subject in need of treatment with the hydroxypropyl methylcellulose.

10. The method of claim 9, wherein it takes 3-4 hours to release 50% of the total amount of hydroxypropyl methylcellulose.

11. A contact lens comprising:
a hydrogel which comprises a three-dimensional network of cross-linked polymer chains; and
at least one ophthalmic drug embedded in the three-dimensional network of cross-linked polymer chains,
wherein the hydrogel comprises a first repeating unit derived from hydroxyethyl methacryalate and a second repeating unit derived from N-vinylpyrrolidone,
wherein the hydrogel comprising the first and second repeating units is configured to swells when absorbing water such that, when the contact lens contacts an aqueous solution having pH in a range between 5.80 and 8.03, the higher the pH of the aqueous solution within the range, the more the hydrogel absorbs water and swell and the more the contact lens releases the at least one ophthalmic drug into the aqueous solution.

12. The contact lens of claim 11, wherein one ophthalmic drug is hydroxypropyl methylcellulose.

13. The contact lens of claim 11, wherein, when being placed in an aqueous solution at pH 8.03, the contact lens is configured to release the at least one ophthalmic drug such that it takes 3-4 hours from beginning to release 50% of the total amount of the at least one ophthalmic drug.

14. A method of making the contact lens of claim 11, the method comprising:
mixing N-vinylpyrrolidone, hydroxyethyl methacrylate and a crosslinking agent to provide a composition;
injecting the composition into a contact lens mold to form the hydrogel therein; and
subsequently loading the at least one ophthalmic drug such that the at least one ophthalmic drug is embedded in the three-dimensional network of cross-linked polymer chains.

15. The method of claim 14, wherein the at least one ophthalmic drug comprises hydroxypropyl methylcellulose.

16. The method of claim 14, wherein, when being placed in an aqueous solution at pH 8.03, the contact lens is configured to release the at least one ophthalmic drug such that it takes 3-4 hours from beginning to release 50% of the total amount of the at least one ophthalmic drug.

17. A method comprising:
providing the contact lens of claim 11; and
placing the contact lens in an eye of a subject in need of treatment with the at least one ophthalmic drug.

18. The method of claim 17, wherein it takes 3-4 hours to release 50% of the total amount of the at least one ophthalmic drug into the eye.

19. A method comprising:
providing the contact lens of claim 11; and
placing the contact lens in an eye of a subject in need of treatment with the hydroxypropyl methylcellulose.

20. The method of claim 19, wherein it takes 3-4 hours to release 50% of the total amount of hydroxypropyl methylcellulose.
